(19) United States
(12) Patent Application Publication
(10) Pub. No.: US 2012/0203350 A1
(43) Pub. Date: Aug. 9, 2012

(54) KNEE JOINT ENDOPROSTHESIS

(75) Inventors: Thomas Hagen, Tuttlingen (DE); Wilhelm Bloemer, Unteruhldingen-Muehlhofen (DE); Volkmar Jansson, Gilching (DE)

(73) Assignee: Aesculap AG, Tuttlingen (DE)

(21) Appl. No.: 13/401,069

(22) Filed: Feb. 21, 2012

Related U.S. Application Data
(63) Continuation of application No. PCT/EP2010/063325, filed on Sep. 10, 2010.

(30) Foreign Application Priority Data
Sep. 10, 2009 (DE) 10 2009 029 360

(51) Int. Cl. A61F 2/38

(52) U.S. Cl. 623/20.22; 623/20.31; 623/20.27

(57) ABSTRACT

In accordance with the invention a knee joint endoprosthesis comprises a femoral component and a meniscal component mounted for movement relative to and on said femoral component. Said femoral component comprises a medial and a lateral condyle having a medial and a lateral condylar surface. Said meniscal component comprises a medial and a lateral joint surface on which the medial and lateral condylar surfaces bear at least partially. Said medial condyle and said medial joint surface are shaped so as to form a rotary joint with a rotary joint center. Moreover, a rolling motion guiding device is provided for defined rolling of the lateral condylar surface and the lateral joint surface on each other along a curved path which is defined in dependence upon an angle of flexion between femoral component and meniscal component and extends around the rotary joint center.

### CROSS-REFERENCE TO RELATED APPLICATIONS

This application is a continuation of international application number PCT/EP2010/063325 filed on Sep. 10, 2010 and claims the benefit of German application number 10 2009 029 360.4 filed on Sep. 10, 2009.

The present disclosure relates to the subject matter disclosed in international application number PCT/EP2010/063325 of Sep. 10, 2010 and German application number 10 2009 029 360.4 of Sep. 10, 2009, which are incorporated herein by reference in their entirety and for all purposes.

### FIELD OF THE INVENTION

The present invention relates to knee joint endoprosthesis generally, and more specifically to a knee joint endoprosthesis comprising a femoral component and a meniscal component mounted for movement relative to and on said femoral component, said femoral component comprising a medial and a lateral condyle having a medial and a lateral condylar surface, said meniscal component comprising a medial and a lateral joint surface on which the medial and lateral condylar surfaces bear at least partially.

### BACKGROUND OF THE INVENTION

Structurally, knee joint endoprostheses of the kind described at the outset are based on a healthy knee joint. Such a knee joint moves during the bending of the knee, also referred to as flexion, more posteriorly on the lateral side than on the medial side and due to the thus generated axial rotation during the bending of the knee joint enables optimum guidance of the patella by the trochlea. The position of the trochlea in relation to the patella has a very strong influence on an optimum redirecting of forces, in particular, on the stretching capability. It is therefore known to design knee joint endoprostheses of the kind described at the outset in the form of uncoupled, bicondylar knee implants with a meniscal component fixed to the tibia or to a tibial component secured to the tibia, which allow the possibility of unforced axial rotation of the tibia in relation to the femur.

In the case of an unfavorable ligament situation or partly missing cruciate ligaments, a posterior displacement of the femoral component is enforced, at least on the lateral side, by the known implant construction. In this way, during flexion, the femur later comes into contact with the meniscal component which, in particular, is made of polyethylene (PE), or with the tibial component, so that theoretically a larger bend or flexion angle between tibia and femur or their longitudinal axes is thereby achieved. The posterior translation is usually brought about by the interaction of a post and a corresponding cam between the femoral component and the meniscal component or via a third condyle. However, it is not possible to thereby achieve a perfect positional alignment of the trochlea, as is the case in the natural, healthy knee joint.

### SUMMARY OF THE INVENTION

In accordance with the invention a knee joint endoprosthesis comprises a femoral component and a meniscal component mounted for movement relative to and on said femoral component. Said femoral component comprises a medial and a lateral condyle having a medial and a lateral condylar surface. Said meniscal component comprises a medial and a lateral joint surface on which the medial and lateral condylar surfaces bear at least partially. Said medial condyle and said medial joint surface are shaped so as to form a rotary joint with a rotary joint center. Moreover, a rolling motion guiding device is provided for defined rolling of the lateral condylar surface and the lateral joint surface on each other along a curved path which is defined in dependence upon an angle of flexion between femoral component and meniscal component and extends around the rotary joint center.

### BRIEF DESCRIPTION OF THE DRAWING FIGURES

The foregoing summary and the following description may be better understood in conjunction with the drawing figures, of which:

FIG. 1 shows an anterior plan view of an implanted knee joint endoprosthesis with the knee joint extended;

FIG. 2 shows an anterior perspective exploded representation of the femoral component and the meniscal component of the knee joint endoprosthesis from FIG. 1 from above;

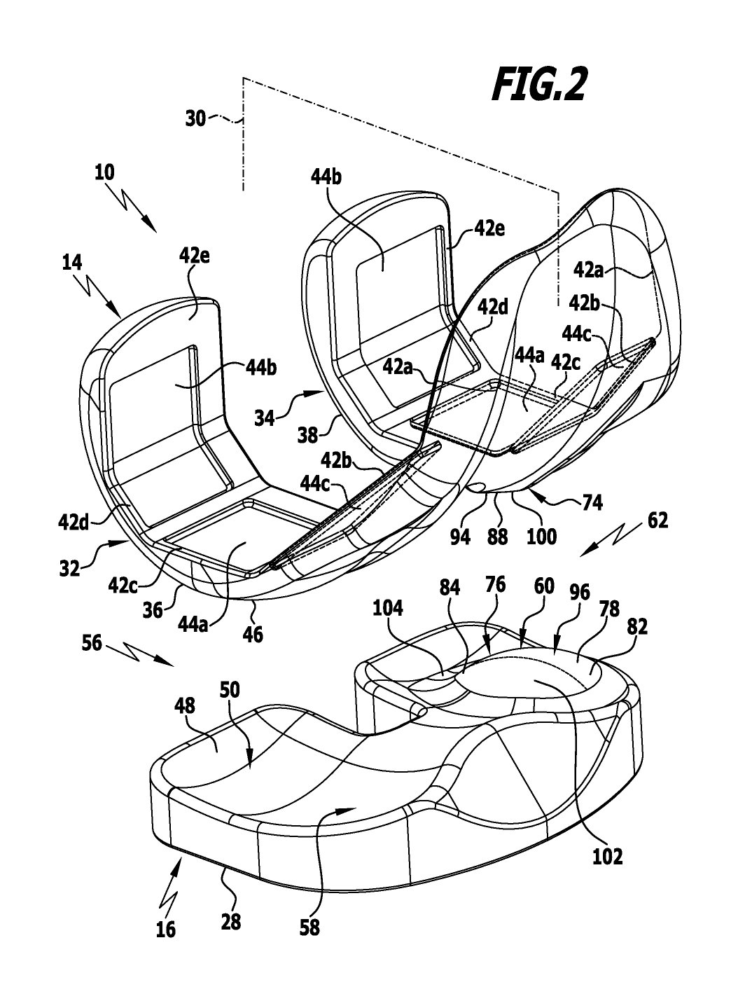

FIG. 3 shows a posterior perspective view in analogy with FIG. 2 from below;

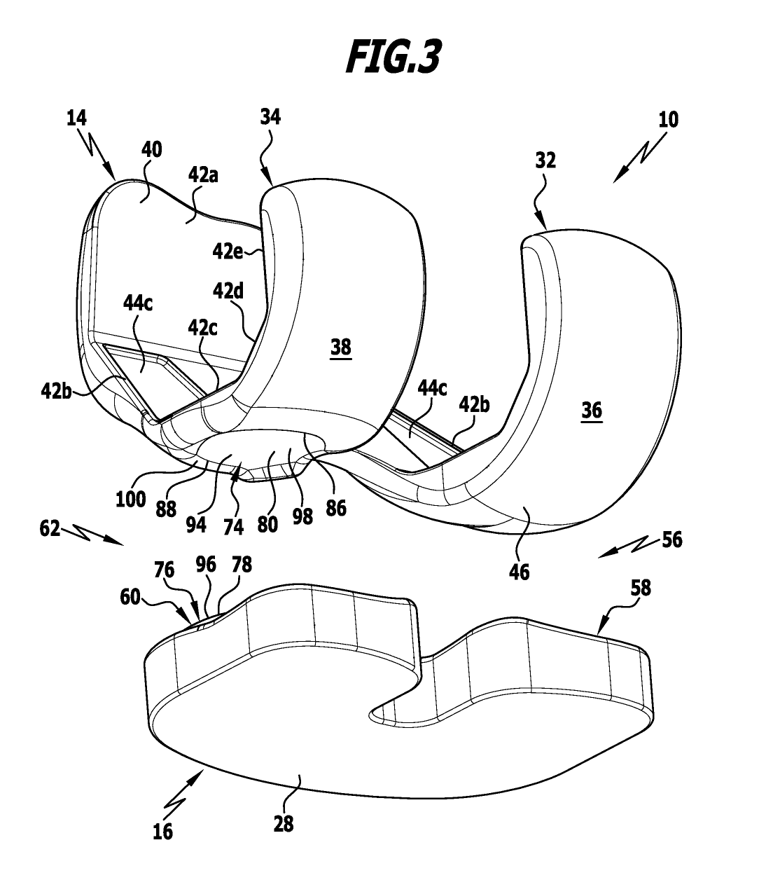

FIG. 4 shows a medial side view of the two components of the knee joint endoprosthesis that are represented in FIGS. 2 and 3;

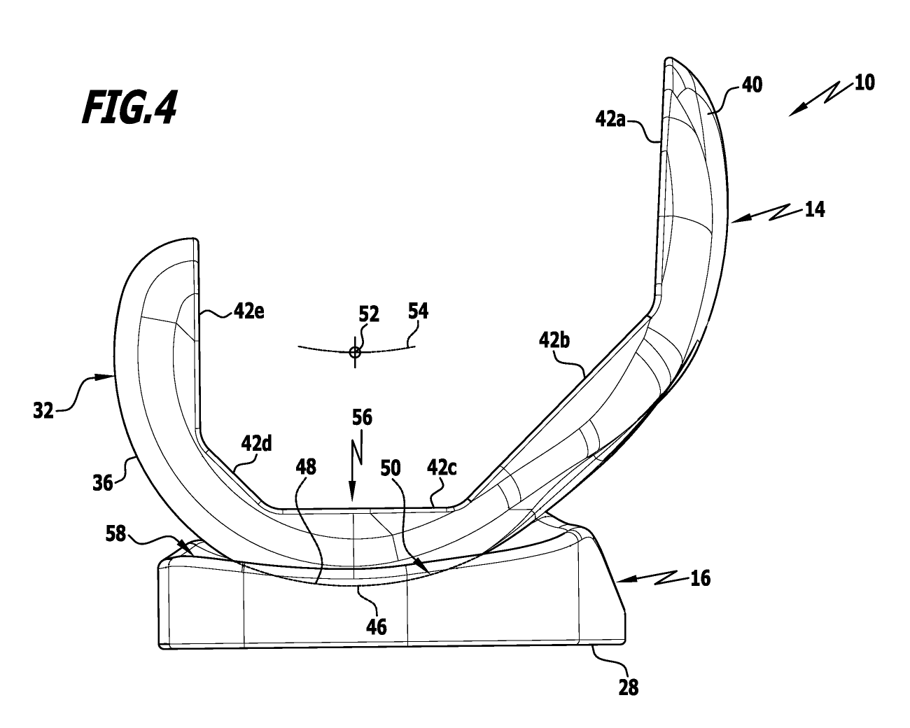

FIG. 5 shows a lateral view in analogy with FIG. 4;

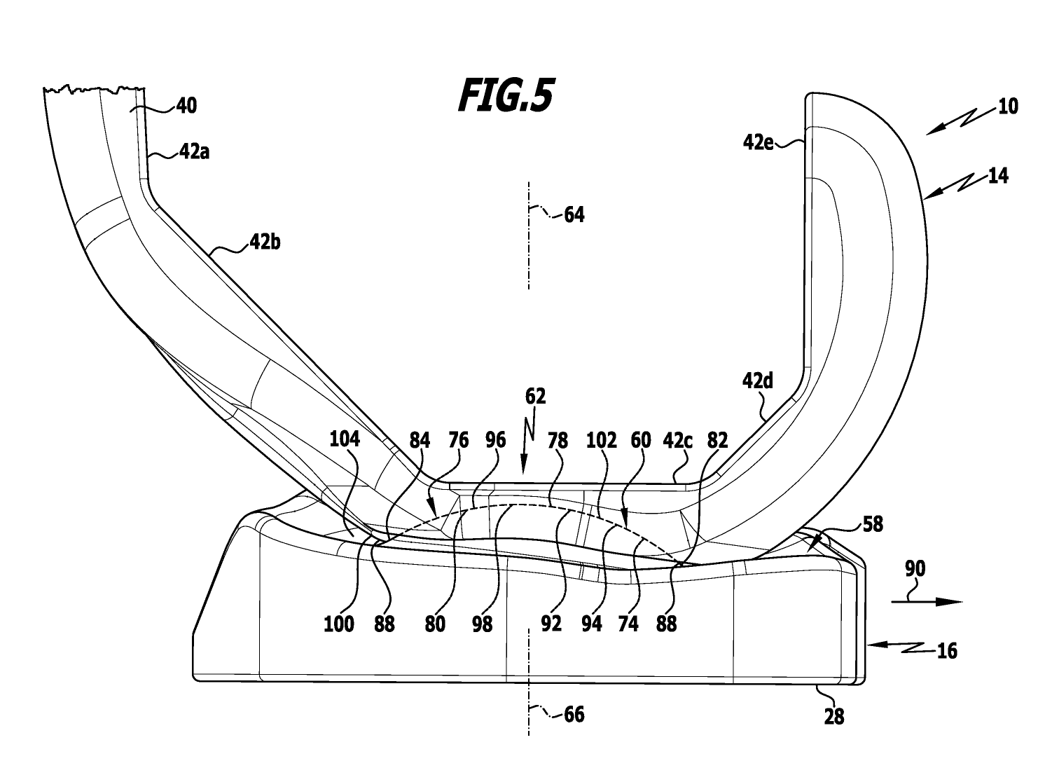

FIG. 6 shows a perspective view of the femoral component and the meniscal component in a flexed position of approximately 90°; and

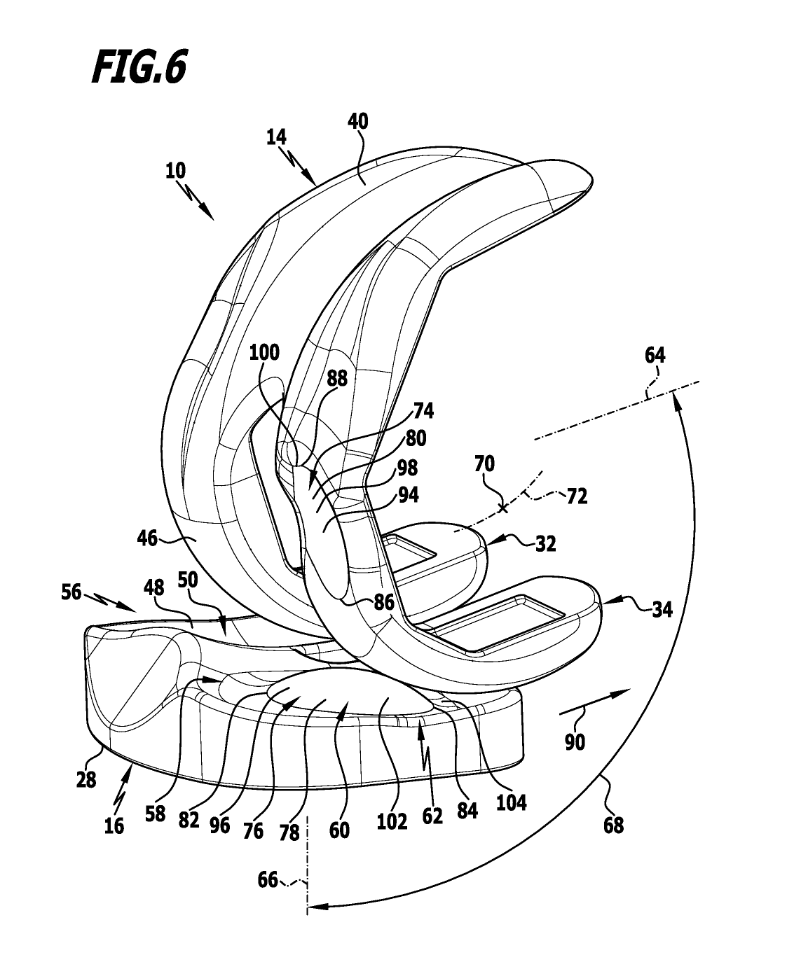

FIG. 7 shows an anterior-lateral view in analogy with FIG. 6.

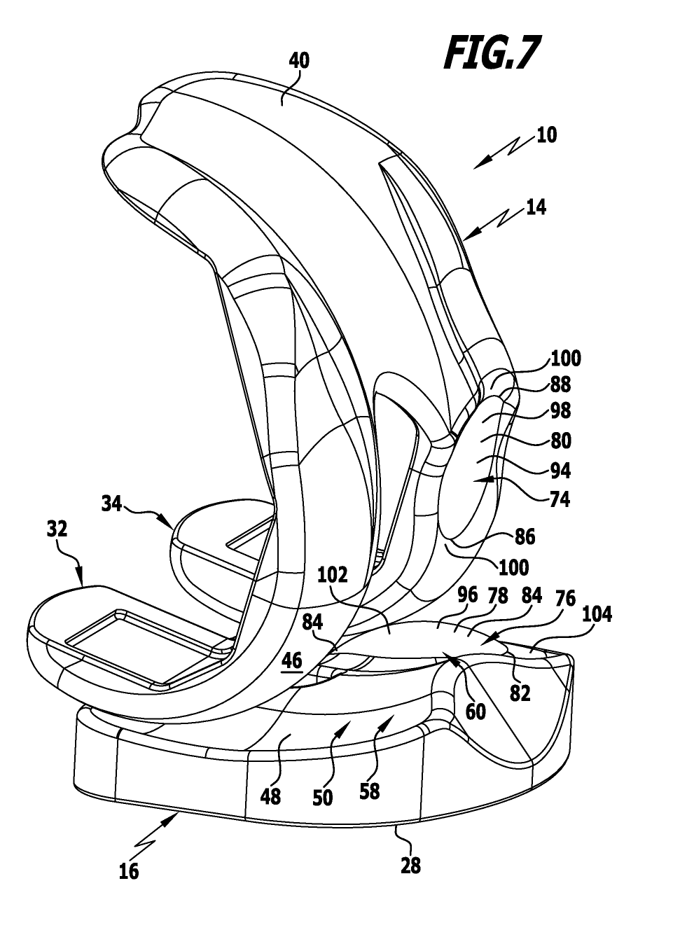

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a knee joint endoprosthesis comprising a femoral component and a meniscal component mounted for movement relative to and on said femoral component, said femoral component comprising a medial and a lateral condyle having a medial and a lateral condylar surface, said meniscal component comprising a medial and a lateral joint surface on which the medial and lateral condylar surfaces bear at least partially, said medial condyle and said medial joint surface being shaped so as to form a rotary joint with a rotary joint center, wherein a rolling motion guiding device is provided for defined rolling of the lateral condylar surface and the lateral joint surface on each other along a curved path which is defined in dependence upon an angle of flexion between femoral component and meniscal component and extends around the rotary joint center.

The knee joint endoprosthesis proposed in accordance with the invention enables a forcibly guided, eccentric rotation of the artificial knee joint formed by the knee joint endoprosthesis upon bending the knee joint. On the medial side, the knee joint endoprosthesis enables rotation about the rotary joint center. On the lateral side, by means of the rolling motion guiding device, during flexion of the knee joint, starting, for example, from the stretching of the patient's leg, no sliding motion is permitted between the femoral component and the meniscal component, but a rolling motion, with the effect that during this the lateral condyle of the femoral component moves somewhat in the posterior direction along the curved path extending around the rotary joint center. The rotation of the knee joint therefore takes place automatically in a forcibly guided manner and is not initiated by soft tissue that is still present. With the proposed knee joint endoprosthesis, it is therefore possible to generate a forcibly guided rotation in the leg axis while moving the joint through the entire range of motion, and, in association with this, to guide the patella in an improved manner in the now anatomically oriented trochlea. A further advantage is the increase in the power of the leg mechanics due to the optimum guidance of the patella now made possible, i.e., improved quadriceps power. Patella complications, in particular, a subluxation or a luxation associated with pain and joint failure are reduced by the improved guidance of the patella. The meniscal component has a significantly higher service life because the post/cam mechanics described at the outset for forced guidance of the femur relative to the tibia posteriorly as a result of the bending can be dispensed with. Furthermore, the risk of breakage or severe wear of the post—not present—is fully eliminated. In addition, the overall size of the meniscal component can be reduced, and consequently, the operating technique also simplified. More individual care of the patient concerned is also possible.

It is advantageous if the rotary joint center is in the form of a joint center which is fixed relative to the femoral component and to the meniscal component. Such a fixed joint center can be formed in a simple way. In addition, better stabilization of the knee joint is thus also possible in the case of very weak or degenerated ligaments.

The rotary joint is preferably in the form of a ball-and-socket joint. A ball-and-socket joint enables in a simple way, in particular, the formation of a fixed joint center.

The structure of the knee joint endoprosthesis is particularly simple if the medial condylar surface comprises a spherical condylar surface area and if the medial joint surface comprises a hollow-spherical joint surface area corresponding to the medial condylar surface. The spherical condylar surface area can thus be mounted directly in or on the hollow-spherical joint surface area, with their radii of curvature preferably being adapted to each other such that a fixed joint center is defined relative to the femoral component and to the meniscal component.

It is advantageous if the fixed joint center is configured so as to exclusively enable a sliding motion of the femoral component and the meniscal component relative to each other. In this way, the femoral component is prevented from being able to move in posterior direction relative to the meniscal component as a result of bending of the knee joint.

In accordance with a further preferred embodiment of the invention, it may, furthermore, be provided that the rotary joint center is in the form of a joint center moving relative to the femoral component and to the meniscal component along a rotary joint center path extending from anterior to posterior in dependence upon an angle of flexion between femoral component and meniscal component. The moving joint center enables, in particular, as a result of bending the knee joint, motion of the femoral component on the medial side to some extent in posterior direction. This may be a translational sliding and/or rolling motion of the femoral component and the meniscal component on each other.

The knee joint endoprosthesis is advantageously so configured that the rotary joint center path extends in a straight line or is convexly curved facing away from the meniscal component in medial direction. The rotary joint center path can be prescribed by corresponding configuration of the medial condylar surface and the medial joint surface, for example, by correspondingly curved medial condylar surface areas and a corresponding medial joint surface area of the meniscal component.

A moving joint center can be formed in a particularly simple way if a radius of curvature of the medial joint surface is larger than a radius of curvature of the medial condylar surface. This preferably involves those surface areas along which the femoral component and the meniscal component bear on each other when the knee joint is being bent, i.e., during flexion, or corresponding stretching, also referred to as extension or extension movement. In this way, motion of the medial condyle of the femoral component relative to the medial joint surface of the meniscal component is enabled upon flexion movement in posterior direction. The meniscal component and the femoral component are preferably so configured that exclusively a sliding motion is enabled between the medial condyle and the medial joint surface.

It is advantageous if the moving joint center is configured so as to enable a superimposed sliding/rolling motion of the femoral component and the meniscal component relative to each other. A defined translational motion of the femoral component relative to the meniscal component as a result of flexion movement of the knee can therefore also be achieved at least partly on the medial side.

In order to simply enable a forced guidance for prescribing a rolling motion of the lateral condyle and the lateral joint surface relative to each other, it is advantageous if the rolling motion guiding device comprises first and second guiding elements interacting with each other, which are formed on the femoral component and on the meniscal component to define a contact surface area moving from anterior to posterior and vice versa around the rotary joint center in dependence upon an angle of flexion between femoral component and meniscal component. A sliding motion between the lateral condyle and the lateral joint surface is fully or essentially fully prevented by the first and second guiding elements. In analogy with the engagement of the teeth of a gear wheel and a gear rack, a rolling motion of the knee joint on the lateral side is thus enforced by the guiding elements. The first and second guiding elements may, however, also be so configured that not an exclusive rolling motion is enforced, but that a sliding motion on the lateral side is also possible, in particular, to a limited extent.

For particularly optimized guidance of the patella in the trochlea it is advantageous if the rolling motion guiding device is configured so as to exclusively enable a rolling motion between the lateral condylar surface and the lateral joint surface. As explained above, this may be achieved, in particular, by the first and second guiding elements being configured so as to engage each other in such a way that, similarly to the engagement of teeth of a gear wheel and a gear rack, they do not allow the parts to slide relative to each other.

The guiding elements can be formed in a particularly simple way if the femoral component comprises at least one first guiding element, and if the meniscal component comprises at least one second guiding element, with the at least one first and one second guiding elements defining first and second guiding element surfaces bearing at least partially on each other. The first and second guiding element surfaces bearing on each other enforce as a result of motion of the knee joint, i.e., for example, upon bending or stretching, a rolling motion of the femoral component and the meniscal component relative to each other on the lateral side.

It is advantageous if the first guiding element surface comprises at least one concave surface area facing in the direction towards the meniscal component. This allows, for example, a corresponding convex surface area of the meniscal component to engage the concave surface area of the femoral component during the desired rolling motion as a result of flexion of the knee joint.

To achieve as good a guidance or interlocking as possible between the femoral component and the meniscal component in order to enforce a rolling motion on the lateral side, it is advantageous if the first guiding element surface comprises two or more concave surface areas which are separated in each case by a convex surface area. These may be arranged relative to one another in a row, which may be of straight-lined or curved shape, behind one another or also partly next to one another or next to one another and behind one another in offset relation to one another.

In order that the first and second guiding element surfaces can interact in a simple way, it is advantageous if the second guiding element surface comprises at least one convex surface area facing in the direction towards the femoral component. The convex surface area can receive, in particular, a concave surface area of the femoral component, in order to enforce a rolling motion of the femoral component and the meniscal component relative to each other.

To enable a defined rolling motion to be prescribed, as far as possible, over a large area, it is advantageous if the second guiding element surface comprises two or more convex surface areas which are separated in each case by a concave surface area.

A particularly small overall size of the meniscal component can be achieved, if, in particular, the first and second guiding elements are formed in the area of the lateral joint surface and the lateral condylar surface. In particular, the first and second guiding elements can be integrated into the lateral joint surface and into the lateral condylar surface, respectively, and, in each case, formed in one piece with these. A guidance directly in the areas where the femoral component and the meniscal component are in contact with each other on the lateral side can thereby be achieved.

The first and second guiding elements preferably comprise first and second guiding element surfaces, which form at least partially the lateral joint surface and the lateral condylar surface. The femoral component and the meniscal component can thereby be guided on each other in those areas in which they are in contact with each other.

The lateral condylar surface advantageously comprises at least one concave surface area facing in the direction towards the meniscal component. Such a concave surface area may, for example, be in the form of a depression in the otherwise preferably predominantly convexly curved femoral joint surface facing away from the femoral component. A corresponding projection protruding convexly and facing away from the meniscal component or an elevation can engage the depression or recess and thus bring about a forced rolling motion upon flexion of the knee joint from a stretched position to any bent position.

In accordance with a further preferred embodiment of the invention, it may be provided that the lateral condylar surface comprises two or more concave surface areas, which are separated in each case by a convex surface area. A two-toothed or multiple-toothed interlocking between the femoral component and the meniscal component can thus be formed in a simple way in order to enforce a rolling motion as a result of flexion movement of the knee joint. It should be noted that the engaging projections and recesses, which define convex and concave surface areas may be of different shape, or, as in a connection between a gear wheel and a gear rack, may be of identical shape.

In order to further simplify and optimize the interaction of the first and second guiding elements, it is advantageous if the lateral joint surface comprises at least one convex surface area facing in the direction towards the femoral component. The convex surface area, which, for example, may form part of an outer surface of a projection, can thus engage in a simple way a corresponding recess, having a concave surface area, in the femoral component.

An optimized interaction of the first and second guiding elements is possible, in particular, when the lateral joint surface comprises two or more convex surface areas which are separated in each case by a concave surface area.

It is, in principle, conceivable to fix the meniscal component directly to the tibia. To facilitate, if need be, exchange of the meniscal component, as a result of wear or damage, it is advantageous if the knee joint endoprosthesis further comprises a tibial component carrying the meniscal component. It is thus possible to first anchor the tibial component in a patient's tibia, for example, with cement or screws, and to then connect it to the meniscal component.

In a large number of knee joint endoprostheses, provision is made to mount the meniscal component and the tibial component so as to be movable on each other. In accordance with a preferred embodiment of the present invention, it is, however, advantageous if the meniscal component and the tibial component are immovably connected to each other. In this way, the meniscal component can be fixed in a defined manner relative to the patient's tibia. The femoral and meniscal components configured for best possible physiological reconstruction of a knee joint enable in the manner described above an at least partly forced rolling motion on each other on the lateral side as a result of flexion movement of the knee.

The femoral component and also the meniscal component can be constructed in a particularly simple and compact way if the femoral component comprises other than the lateral condyle and the medial condyle no further condyle. The femoral component then exclusively comprises a lateral condyle and a medial condyle. Furthermore, in the knee joint endoprosthesis, there is preferably no projection provided in the form of a post or the like on the meniscal component and/or on the tibial component, which serves to guide the meniscal component and the femoral component during bending movement of the knee. In this way, the overall size of the meniscal component can be simply and safely minimized.

The knee joint endoprosthesis can be produced in a simple way, and, in addition, its stability increased, if the femoral component and/or the meniscal component and/or the tibial component are each constructed in one piece. In particular, the femoral component and the tibial component can be formed from an implant material, for example, an implant steel or titanium, the meniscal component from an abrasion-resistant plastic material, for example, polyethylene or polyethylene of high density and with a high molecular weight.

In accordance with a further preferred embodiment of the invention, it may be advantageous if the femoral component and/or the tibial component are in the form of modular prosthesis parts. Both the femoral component and the tibial component can thus be individually adapted to the respective physiology of the patient. For example, both the femoral component and the tibial component can comprise shafts which can be inserted in corresponding cavities of femur and tibia and fixed therein, for example, with bone cement or bone screws. Furthermore, the shafts can be detachably connectable to further constituents of the femoral component and the tibial component, respectively, so that, as required, the femoral component and the tibial component can be individually assembled by an operating surgeon during a surgical procedure in order to enable best possible adaptation of the knee joint endoprosthesis to the patient's physiology.

In principle, it is conceivable to produce the femoral component and the meniscal component from identical materials. In order to minimize friction and, consequently, wear due to abrasion, it is advantageous if the femoral component and the meniscal component are made from different materials. The femoral component and the tibial component are advantageously produced from identical materials. It is advantageous if the femoral component is made of a more abrasion-resistant material than the meniscal component. In this way, wear of the knee joint endoprosthesis can be directed to that part which, if need be, is easier to exchange.

The following description of preferred embodiments of the invention serves in conjunction with the drawings for a more detailed explanation.

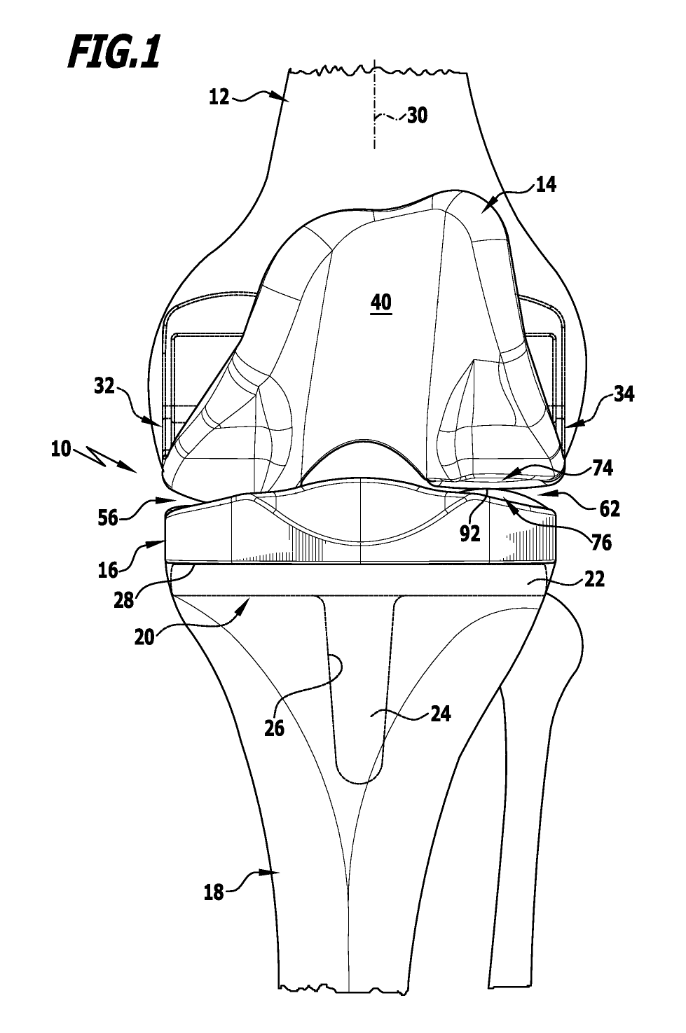

FIG. 1 shows a knee joint endoprosthesis generally designated by reference numeral 10. It comprises a bicondylar femoral component 14 fixable to a femur 12, and a meniscal component 16 mounted so as to be movable relative to and on the femoral component 14.

The meniscal component 16 may, optionally, be directly connected to a partially resected tibia 18 of a patient, for example, by screws or cementing. It may, alternatively, be immovably fixed to a tibial component 20, shown in dashed lines in FIG. 1, of the knee joint endoprosthesis 10.

The tibial component 20 comprises a plate 22 and a shaft 24 protruding substantially transversely from the plate 22. The shaft 24 is fixed in a correspondingly shaped cavity 26 of the tibia 18, for example, by means of bone cement or bone screws, not shown. The meniscal component 16 is fixed immovably to the tibial component 20 by a connecting device, not shown. The connecting device may, in particular, be in the form of a latching or snap connection with latching or snap elements which engage one another. The latching or snap elements are formed on the plate 22 and on an underside 28 which is otherwise of flat construction. Alternatively, the connecting device may comprise fastening elements, for example, screws, with which the meniscal component can be immovably fixed to the tibial component 20.

The femoral component 14, as well as the meniscal component 16, is of asymmetrical configuration in relation to a sagittal plane 30 and comprises two condyles, namely a medial condyle 32 and a lateral condyle 34, which have a medial condylar surface 36 and a lateral condylar surface 38. The condylar surfaces 36 and 38 face away from the femoral component 14 in the direction towards the meniscal component 16.

The condyles 32 and 34 are spaced from each other in lateral-medial direction and are integrally connected to each other by a connecting element 40 at their anterior ends. Bearing surfaces 42a, 42b, 42c, 42d and 42e facing away from the meniscal component 16 for positioning the meniscal component on a correspondingly prepared femur 12 are respectively inclined at 45° relative to each other. There is formed in the bearing surfaces 42b and 42c a first recess 44a, which has a constant depth in each of the bearing surfaces 42b and 42c, but does not extend over the entire surface defined by the two bearing surfaces 42b and 42c. A further recess 44b, starting from the bearing surfaces 42d and 42e, is formed in the condyles 32 and 34. The bearing surfaces 42a to 42e together with the recesses 44a and 44b of the two condyles 32 and 34 serve to fix, preferably with bone cement, the femoral component 14 to a femur which has previously been partially resected in accordance with an inner contour of the femoral component 14, which is prescribed by the bearing surfaces 42a to 42e.

The condylar surfaces 36 and 38 are of different shape. The medial condylar surface 36 has a spherical condylar surface area 46, which is exclusively convexly curved and faces away from the femoral component 14. A radius of curvature of the spherical condylar surface area 46 is somewhat smaller than a hollow-spherical joint surface area 48 of a medial joint surface 50 of the meniscal component 16, on which the medial condylar surface 36 partly bears with the spherical condylar surface area 46. Optionally, the radii of curvature of the spherical condylar surface area 46 and the hollow-spherical joint surface area 48 may be identical, so that a rotary joint center 52 of a ball-and-socket joint is defined. The rotary joint center 52 may be in the form of a fixed joint center if the radius of the spherical condylar surface area 46 and the radius of the hollow-spherical joint surface area 48 are identical. If the tub-shaped, hollow-spherical joint surface area 48 is less curved than the spherical condylar surface area 46, the rotary joint center 52 may move along a rotary joint center path 54 from a maximum anterior position, with the knee joint extended, in the posterior direction towards a maximum posterior end position in which the knee joint is bent at a maximum. Altogether, the medial condyle 32 then also forms with the medial joint surface a rotary joint 56.

The medial joint surface 50 forms part of an upper side 58 of the meniscal component 16, which further comprises a lateral joint surface 60, which is configured so as to interact with the lateral condylar surface 38 in the manner described below.

As mentioned at the outset, knee joint endoprostheses with an eccentric rotary joint 56 as described above are known. To achieve a defined rolling, preferably only of the lateral condyle 34 and the lateral joint surface 60, as a result of bending the knee joint, the knee joint endoprosthesis 10 comprises a rolling motion guiding device 62. It serves to enforce a defined rolling motion of the lateral condylar surface 38 and the lateral joint surface 60 on each other along a curved path 72, which is defined in dependence upon an angle of flexion 68 of the knee joint, i.e., between a longitudinal axis 64 of the femur and a longitudinal axis 66 of the tibia, and extends around the rotary joint center 52. This has the consequence that a lateral joint center 70 moves along a path 72 in posterior direction as a result of the bending. If the joint center 52 is a fixed joint center, the path 72 extends around the fixed joint center 52. This results from the rotary joint 56 being in this case a ball-and-socket joint. The forced rolling motion is achieved by the special design of the rolling motion guiding device 62, which, in principle, corresponds constructionally to a gear wheel and a gear rack, which are moved relative to each other. First guiding elements 74 correspond to teeth of a gear wheel and second guiding elements 76 on the meniscal component 16 to teeth of the gear rack. The first and second guiding elements 74 and 76 are in the form of depressions 78 and 80 and elevations 82 and 84 on the meniscal component 16 and 86 and 88 on the femoral component 14. They are arranged in such a way that upon bending the knee, the elevations 82, 84, 86 and 88 sink into the correspondingly shaped depressions 78 and 80. As a result of the special design of the depressions 78 and 80 and the elevations 82, 84, 86 and 88 an interlocking is achieved, which prevents sliding motion of the knee joint endoprosthesis 10 on the lateral side, i.e., between the lateral condylar surface 38 and the lateral joint surface 60. In addition, the first and second guiding elements 74 and 76 are preferably formed concentrically with the rotary joint center 52, so that a rotation is enforced about the rotary joint center 52 on the medial side and a superimposed rotational/rolling motion is enforced about the rotary joint center 52 on the lateral side. During this, the joint center 70, which is essentially defined by the lateral condyle 34, moves along the path 72, as the angle of flexion 68 increases, in the direction of arrow 90 in posterior direction.

In the case of a moving rotary joint center 52, the rotary joint center path 54 is, depending on the configuration of the medial joint surface 50 and the associated medial condylar surface 36, either straight-lined or convexly curved facing away from the meniscal component 16 in medial direction.

In an analogous manner, a contact surface area 92 also moves in posterior direction, the contact surface area 92 being defined by the lateral condylar surface 38 and the lateral joint surface 60 bearing on each other.

The first and second guiding elements 74 and 76 define first and second guiding element surfaces 94 and 96 for guiding the femoral component 14 and the meniscal component 16 relative to each other. The first guiding element surface 94 has at least one concave surface area 98, which is bounded at the sides by convex surface areas 100. In an analogous manner, the second guiding element surface 96 has at least one convex surface area 102, preferably two convex surface areas 102, facing in the direction towards the femoral component 14, which are preferably separated from each other by a concave surface area 104.

The first and second guiding elements 74, 76 are formed in the region of the lateral joint surface 60 and the lateral condylar surface 38, which results in a particularly small overall size of the meniscal component 16. They therefore form at least partly the lateral joint surface 60 and the lateral condylar surface 38.

The femoral component 14 and the tibial component 20 may optionally be in the form of modular prosthesis parts, which may be made up of a plurality of components which are connected to one another and, if need be, are exchangeable.

The described implant design, which, upon moving the leg, generates a forcibly guided axial rotation about the eccentric rotary joint center 52, optionally also superimposed on the medial side with a posterior translation, and therefore enables a defined alignment of the trochlea, contributes significantly towards maintaining the physiological kinematics of the knee joint. This requires neither further guiding elements between the tibial component 20 and the femoral component 14, known, for example, in the form of posts and corresponding cams, nor further condyles on the femoral component, i.e., in particular, no third condyle.

The femoral component 14 and the tibial component 20 are preferably made of an implant steel or titanium or a titanium alloy, the meniscal component 16 preferably of an abrasion-resistant plastic material, for example, polyethylene (PE).

1. Knee joint endoprosthesis comprising a femoral component and a meniscal component mounted for movement relative to and on said femoral component, said femoral component comprising a medial and a lateral condyle having a medial and a lateral condylar surface, said meniscal component comprising a medial and a lateral joint surface on which the medial and lateral condylar surfaces bear at least partially, said medial condyle and said medial joint surface being shaped so as to form a rotary joint with a rotary joint center, wherein a rolling motion guiding device is provided for defined rolling of the lateral condylar surface and the lateral joint surface on each other along a curved path which is defined in dependence upon an angle of flexion between femoral component and meniscal component and extends around the rotary joint center.

2. Knee joint endoprosthesis in accordance with claim 1, wherein the rotary joint center is in the form of a joint center which is fixed relative to the femoral component and to the meniscal component.

3. Knee joint endoprosthesis in accordance with claim 1, wherein the rotary joint is in the form of a ball-and-socket joint.

4. Knee joint endoprosthesis in accordance with claim 3, wherein the medial condylar surface comprises a spherical condylar surface area, and the medial joint surface comprises a hollow-spherical joint surface area corresponding to the medial condylar surface.

5. Knee joint endoprosthesis in accordance with claim 2, wherein the fixed joint center is configured so as to exclusively enable a sliding motion of the femoral component and the meniscal component relative to each other.

6. Knee joint endoprosthesis in accordance with claim 1, wherein the rotary joint center is in the form of a joint center moving relative to the femoral component and to the meniscal component along a rotary joint center path extending from anterior to posterior in dependence upon an angle of flexion between femoral component and meniscal component.

7. Knee joint endoprosthesis in accordance with claim 6, wherein the rotary joint center path extends in a straight line or is convexly curved facing away from the meniscal component in medial direction.

8. Knee joint endoprosthesis in accordance with claim 6, wherein a radius of curvature of the medial joint surface is larger than a radius of curvature of the medial condylar surface.

9. Knee joint endoprosthesis in accordance with claim 6, wherein the moving joint center is configured so as to enable a superimposed sliding/rolling motion of the femoral component and the meniscal component relative to each other.

10. Knee joint endoprosthesis in accordance with claim 1, wherein the rolling motion guiding device comprises first and second guiding elements interacting with each other, which are formed on the femoral component and on the meniscal component to define a contact surface area moving from anterior to posterior and vice versa around the rotary joint center in dependence upon an angle of flexion between femoral component and meniscal component.

11. Knee joint endoprosthesis in accordance with claim 1, wherein the rolling motion guiding device is configured so as to exclusively enable a rolling motion between the lateral condylar surface and the lateral joint surface.

12. Knee joint endoprosthesis in accordance with claim 10, wherein the femoral component comprises at least one first guiding element, and the meniscal component comprises at least one second guiding element, said at least one first and one second guiding elements defining first and second guiding element surfaces bearing at least partially on each other.

13. Knee joint endoprosthesis in accordance with claim 12, wherein the first guiding element surface comprises at least one concave surface area facing in the direction towards the meniscal component.

14. Knee joint endoprosthesis in accordance with claim 13, wherein the first guiding element surface comprises two or more concave surface areas which are separated in each case by a convex surface area.

15. Knee joint endoprosthesis in accordance with claim 12, wherein the second guiding element surface comprises at least one convex surface area facing in the direction towards the femoral component.

16. Knee joint endoprosthesis in accordance with claim 15, wherein the second guiding element surface comprises two or more convex surface areas which are separated in each case by a concave surface area.

17. Knee joint endoprosthesis in accordance with claim 12, wherein the first and second guiding elements are formed in the area of the lateral joint surface and the lateral condylar surface.

18. Knee joint endoprosthesis in accordance with claim 12, wherein the first and second guiding elements comprise first and second guiding element surfaces, which form at least partially the lateral joint surface and the lateral condylar surface.

19. Knee joint endoprosthesis in accordance with claim 1, wherein the lateral condylar surface comprises at least one concave surface area facing in the direction towards the meniscal component.

20. Knee joint endoprosthesis in accordance with claim 19, wherein the lateral condylar surface comprises two or more concave surface areas, which are separated in each case by a convex surface area.

21. Knee joint endoprosthesis in accordance with claim 1, wherein the lateral joint surface comprises at least one convex surface area facing in the direction towards the femoral component.

22. Knee joint endoprosthesis in accordance with claim 21, wherein the lateral joint surface comprises two or more convex surface areas which are separated in each case by a concave surface area.

23. Knee joint endoprosthesis in accordance with claim 1, further comprising a tibial component carrying the meniscal component.

24. Knee joint endoprosthesis in accordance with claim 1, wherein the femoral component comprises other than the lateral condyle and the medial condyle no further condyle.

25. Knee joint endoprosthesis in accordance with claim 1, wherein the femoral component and at least one of the meniscal component and the tibial component are each formed in one piece.
